(19) United States
(12) Patent Application Publication
(10) Pub. No.: US 2001/0046300 A1
(43) Pub. Date: Nov. 29, 2001

(54) Offline active control of automotive noise

(76) Inventor: Ian R. McLean, Chatham (CA)

Correspondence Address: Elsa Keller, SIEMENS CORPORATION, 186 Wood Avenue South, Iselin, NJ, 08830, US

(21) Appl. No.: 09/779,725

(22) Filed: Feb. 8, 2001

Related U.S. Application Data
(60) Provisional application No. 60/198,077, filed on Apr. 17, 2000.

(51) Int. Cl. A61F 11/06; G10K 11/16; H03B 29/00; H04B 1/00

(52) U.S. Cl. 381/71.4; 381/71.5; 381/71.9; 381/86

(57) ABSTRACT

The active noise attenuation system comprises a speaker, a control unit in communication with the speaker, and a memory unit in communication with the control unit storing cancellation waveform data. The system uses the stored cancellation waveform data to create a cancellation waveform through a speaker in proximity to an air induction system to thereby attenuate engine noise. Sensors serve to trigger the release of the cancellation waveform as well as to affect the form of the cancellation waveform to ensure noise attenuation.

This application claims priority to Provisional Patent Application Ser. No.

60/198,077 filed Apr. 17, 2000.

### BACKGROUND OF THE INVENTION

This invention relates to an active control for automotive induction noise.

Manufacturers have employed active and passive methods to reduce engine noise within the passenger compartment. Such noise may travel from the engine and through the air induction system.

Efforts have been made to reduce the amount of engine noise traveling through the air induction system. These efforts include the use of passive devices such as expansion chambers and Helmholtz resonators. Active devices involving anti-noise generators have also been proposed. These systems use a speaker that generates a sound that is out of phase with the engine noise to cancel the noise. This cancellation signal is generated in proximity to the air induction system.

In one such system, the cancellation signal is generated in real time by a digital signal processor based on detected noise levels. Such a system requires a microphone to detect the current engine noise level and a reference signal such as an engine tachometer signal. Based on the signal from the microphone as well as the reference signal, a cancellation signal is created and passed through an audio amplifier to the speaker located in proximity to the air induction system.

Several drawbacks exist to the real time measurement of engine noise and creation of its corresponding cancellation signal. First, a digital signal processor controller is more expensive than a microprocessor based controller. Second, a digital model of the acoustical-mechanical-electrical transmission path of the cancellation signal is required for the stable operation of the digital signal processor based controller. Any change in the physical configuration of the elements included in the signal transmission path will result in the speaker generating loud and annoying noise due to the instabilities arising from the poor modeling of the transmission path. Third, the degree of noise cancellation using real time control is limited during engine acceleration since the required cancellation signal must be generated fast enough to track the change in engine noise generated as the engine speed changes. Finally, real time control requires an error microphone, which must be located such that the ambient vehicle noise does not overwhelm the noise radiating from the annular intake/speaker.

A need therefore exists for a means of creating a cancellation signal using an inexpensive microprocessor based controller to eliminates these problems.

### SUMMARY OF THE INVENTION

In a disclosed embodiment of this invention, the active noise attenuation system comprises a speaker, a control unit in communication with the speaker, and a memory unit in communication with the control unit that stores data relating to the cancellation signal. The cancellation signal is preferably related to engine data such as a particular engine speed. Generally, there is a proportional relationship between such data and engine noise. Because the cancellation signal is stored in memory, the system need not calculate the cancellation signal in real time.

In operation in a vehicle, the speaker is disposed about the air induction system. A sensor communicates with the control unit to trigger the recall from memory of the appropriate cancellation signal necessary to attenuate engine noise at the particular engine speed. In such a configuration, a sensor detects the engine speed and then communicates this speed to the control unit. The control unit then retrieves from the memory unit the appropriate cancellation waveform and then projects this waveform through the speaker to attenuate engine noise. An environmental sensor, such as an air temperature, pressure, or humidity sensor, may serve as input to the control unit to affect the particular form of the cancellation signal.

An important element of this system is the creation and storage of the cancellation signal in the memory unit. While the system normally operates offline, the cancellation signal data for the system is generated in real time. First, engine noise associated with a particular speed is sensed. The cancellation signal needed to attenuate this noise is then determined and then recorded with the particular engine speed. Finally, the cancellation signal data is stored in the memory unit for later recall by the control unit.

By storing the cancellation signal data in the memory unit, the cancellation signal is not determined in real time but instead recalled from the memory unit by the control unit. The response time for such a system is faster than systems currently available. Moreover, only a microprocessor rather than a digital signal processor is required for this system. While a microphone is used to sense and aid in the collection of the cancellation signals, in operation in the vehicle, the system requires no microphone. The information may be determined experimentally for a model of a particular vehicle style, and then utilized and programmed into the control for each vehicle made according to that model. Alternatively, the control could be somewhat more complex, and would be stored on the actual individual vehicle.

### BRIEF DESCRIPTION OF THE DRAWINGS

The various features and advantages of this invention will become apparent to those skilled in the art from the following detailed description of the currently preferred embodiment. The drawings that accompany the detailed description can be briefly described as follows:

FIG. 1 shows an embodiment of the invention including speaker, control unit, and memory unit.

FIG. 2 shows the embodiment of the invention of FIG. 1 in its environment represented schematically.

FIG. 3 shows the means by which the cancellation waveform data is generated and stored.

FIG. 4 is a graph of the scaling factor used to modify the cancellation waveform of the invention.

### DETAILED DESCRIPTION OF THE PREFERRED EMBODIMENT

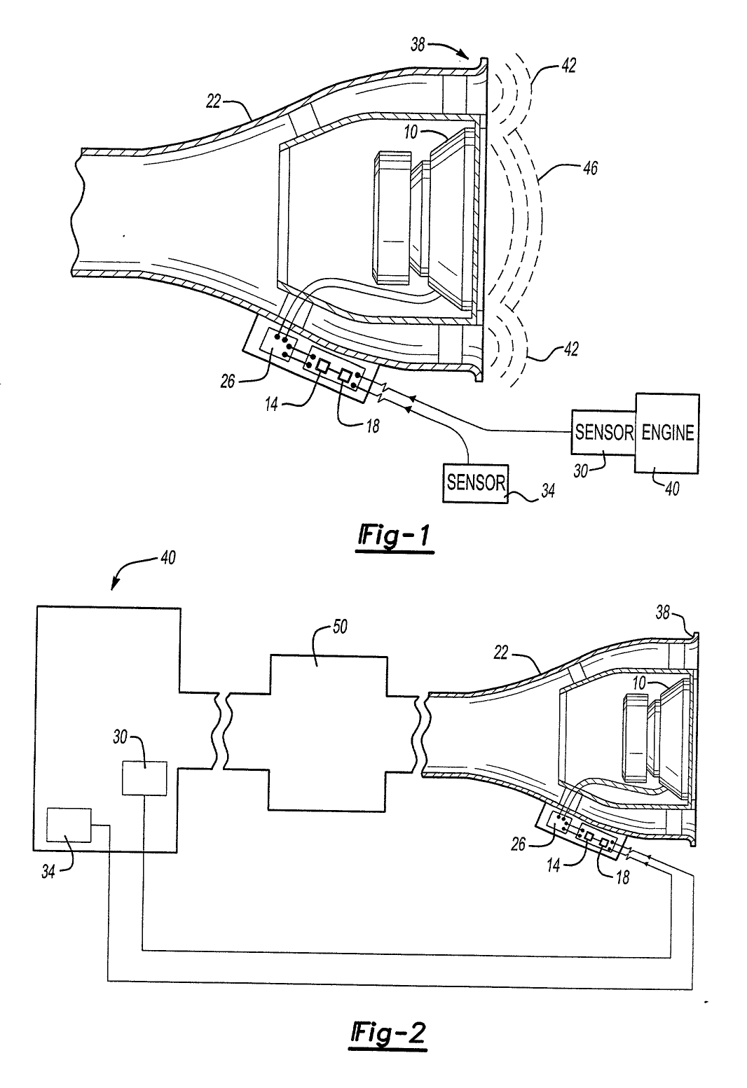

FIG. 1 shows a cross-sectional representation of the invention. Shown are speaker 10, control unit 14, and memory unit 18 within air duct body 22 of an air induction system of a vehicle. As known, the air induction system supplies air to an engine in a manner well-known and not illustrated here. Speaker is an axially symmetric speaker supported to minimize airflow restriction through air induction system. Control unit 14 includes a microprocessor and may include analog to digital converter as well as a digital to analog converter. Control unit 14 is in communication with memory unit 18 as well as speaker 10. Amplifier 26 serves to amplify any signal from control unit 14 to speaker 10.

Memory unit 18 in communication with control unit 14 stores cancellation waveform data. This cancellation waveform data comprises the data necessary to attenuate noise that is preloaded into memory unit 18. Such data may include the actual attenuating waveforms themselves, scaled versions of these waveforms, or their characteristics all organized in a manner for retrieval by control unit 14. Preferably, the cancellation waveform data comprises at least one cancellation waveform related with engine data such as engine speed.

Also shown are two sensors 30 and 34 in communication with control unit 14. One sensor 30 may detect the engine speed while the other sensor may be a throttle position sensor 34. As will be explained in detail, the sensor that detects engine speed 30 provides the timing for the release of the cancellation waveform while the throttle position sensor 34 determines the scaling factor for the amplitude of the cancellation waveform. One of ordinary skill in the art could employ other sensors to inform control unit 14 to optimize noise attenuation. Indeed, such sensors may be environmental sensors that sense air temperature, humidity, and pressure. These environmental conditions, especially air temperature, may impact noise attenuation by the cancellation waveform and may therefore be considered.

As illustrated by FIG. 1 and FIG. 2, speaker 10 is supported within air duct body 22 at mouth 38 as known in the art. In operation, engine noise 42 from engine 40 (shown schematically) has traveled from engine 40 though air duct body 22. Since speaker 10 is co-axially mounted and in the same plane as mouth 38, both the radiated engine noise and the cancellation waveform 46 radiating from speaker 10 share a common location thereby minimizing engine noise 42.

Sensor 30 detects engine speed and communicates with control unit 14. The speed of engine 40 may be computed by control unit 14 from sensor 30. As an example, sensor 30 may emit one pulse every two engine revolutions. Generally, control unit 14 preferably receives a signal from sensor 30 at about half the engine speed. Of course, other ways of identifying engine speed may be used. Based on engine speed, control unit 14 selects the appropriate cancellation waveform 46 from memory unit 18 and determines the amplification necessary to attenuate engine noise based on the size of the throttle opening according to the throttle position sensor 34. Cancellation waveform 46 has a period corresponding to two engine revolutions to match period of engine noise 42 Sensor 34, a throttle position sensor, determines the size of the throttle opening and communicates the throttle position to control unit 14. The amplitude of cancellation waveform 46 is scaled appropriately (a scaling factor of 1 representing a completely open throttle opening while 0 represents a completely closed throttle opening) by control unit 14 through amplifier 26 and then propagated out by speaker 10. Cancellation waveform 46 is out of phase with engine noise 42, preferably 180 degrees out of phase. A ring buffer may be employed in the control to continue the cancellation waveform 46 until engine speed, throttle position, or any other sensed condition changes in the system. In this way, cancellation waveform will continuously serve to attenuate engine noise until conditions change.

It is important to note that the retrieval of cancellation waveform 46 from memory unit 18 take little time to ensure instantaneous response of the system. Even so, the short delay is preferably compensated for the phase of cancellation waveform 46 to accommodate for the delay and ensure optimal wave cancellation. The compensation occurs by delaying cancellation waveform a small time T which is slightly longer than the time required by control unit 14 to retrieve and scale cancellation waveform 46. The time required by the control unit to retrieve and scale the waveform may be determined experimentally based upon the system and then the time could be programmed into the control.

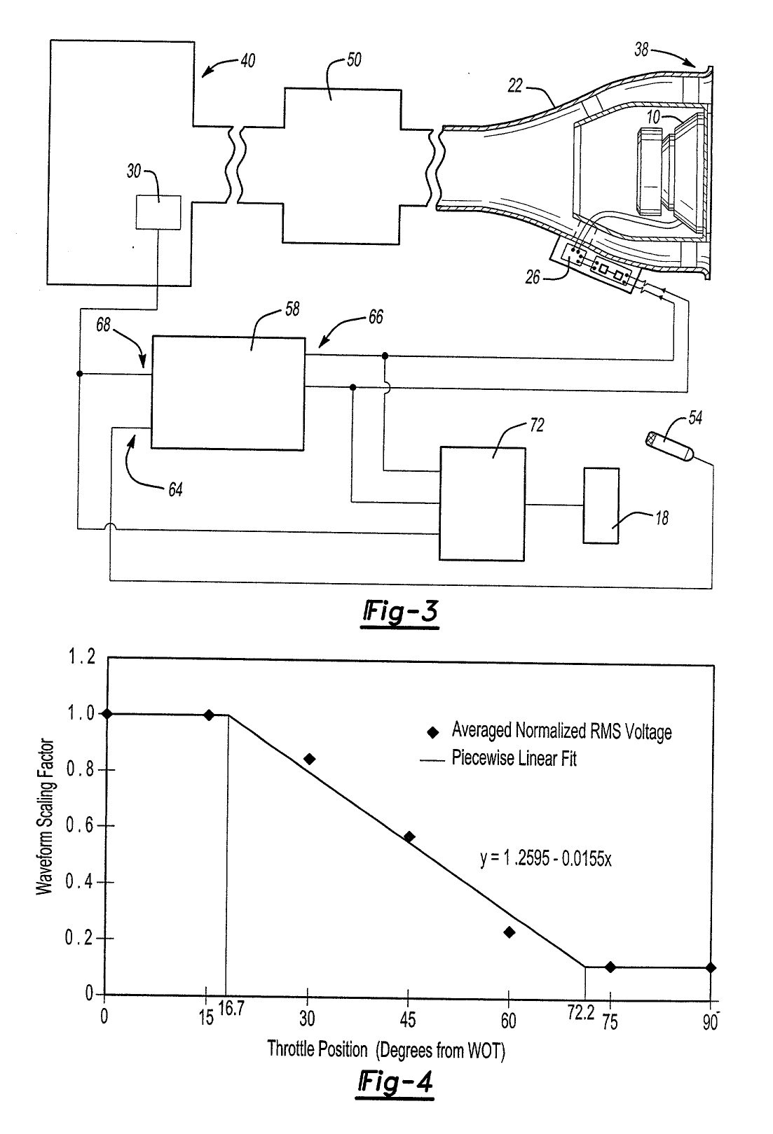

For each vehicle, cancellation waveforms stored in memory unit 18 are generated and stored using a real time system as shown in FIG. 3. Speaker 10 is disposed in mouth 38 of air duct body 22 of air induction system with microphone 54 in close proximity. As known in the prior art, amplifier 26 is connected to real-time digital signal processor controller 58 with analog-to-digital inputs 64 and 68 from microphone 54 and engine speed sensor 30, respectively. Real-time digital signal processor controller 58 also has digital to analog output 72 to computer 76. The embodiment of FIG. 3 generates the cancellation waveform data for every engine speed of engine 40 in real time by a real-time digital signal processor controller 58 as already known in the art. The cancellation waveform data is collected during a slow acceleration of the engine from idle to redline at wide-open throttle. A high-resolution engine speed sensor 30 such as a high-resolution tachometer is employed. The signal from sensor 30 is at least 60 pulses per engine revolution. Also, the signal from microphone 54 is sampled by the real-time digital signal processor controller 58 through analog-to-digital input 64. As known in the art, the real-time cancellation waveform data—the cancellation waveform for each engine speed—is created by this arrangement and communicated to computer 72, which stores this information in memory unit 18 such as an EPROM. Memory unit 18 is subsequently inserted into the system of FIGS. 1 and 2 to permit reference by control unit 14 of data during vehicle operation.

The system of FIG. 3 also determines the scaling factor used to modify the amplitude of the generated cancellation waveform. This scaling factor is determined by operating the real-time digital signal processor controller 58 and configuration of FIG. 3 for each degree of throttle opening and determining the cancellation waveform for each degree of opening. The degree of change of the amplitude of the cancellation waveform needed to attenuate engine noise over the degrees of throttle opening reflects the scaling factor. The scaling factor will vary from engine to engine and vehicle to vehicle. The scaling factor is stored in memory unit 18 for use by control unit 14 to determine the amplitude of cancellation waveform depending on the size of the throttle opening.

The scaling factor for a particular engine and vehicle is shown in FIG. 4. The waveform scaling factor is plotted against throttle position in degrees from wide open (WOT). From 0 degrees to about 16 degrees (open throttle) from throttle wide open, the cancellation waveform need not be scaled down for this particular engine and vehicle. Also, from about 72 to about 90 degrees (closed throttle) from throttle wide open, the cancellation waveform is scaled down significantly. Between about 16 degrees to about 72 degrees, the scaling factor is linear. The scaling factor will vary for each engine and vehicle.

The system of FIG. 3 may be utilized per model type of vehicle to generate information such as that shown in FIG. 4 for a particular vehicle style carrying a particular engine. Then, this information can be stored into each vehicle made according to that model. While more complex, the system could be incorporated into each actual vehicle which would generate the information for the particular vehicle. Further, as mentioned above, sensors sensing environmental factors may also be incorporated into the information such as shown in FIG. 4. Again, while this information would be more complex to generate, store and utilize, it would also provide more effective cancellation of noise. The information tied to particular environmental conditions can be gathered in a similar fashion to that explained above with regard to FIG. 3. Varying environmental conditions can be changed under a control setting, and the resulting information stored. Further, the structure and processes for positioning mounting and operating this speaker may be as known. This invention relates to the generation of a preferred waveform for cancellation of condition.

The aforementioned description is exemplary rather then limiting. Many modifications and variations of the present invention are possible in light of the above teachings. The preferred embodiments of this invention have been disclosed. However, one of ordinary skill in the art would recognize that certain modifications would come within the scope of this invention. Hence, within the scope of the appended claims, the invention may be practiced otherwise than as specifically described. For this reason the following claims should be studied to determine the true scope and content of this invention.

What is claimed is:

1. A noise attenuation system comprising:
a speaker;
a control unit in communication with said speaker; and
a memory unit in communication with said control unit storing cancellation waveform data related to a system condition.

2. The active noise attenuation system of claim 1 wherein said system condition is engine data.

3. The active noise attenuation system of claim 2 wherein said engine data is engine speed.

4. The active noise attenuation system of claim 1 further including at least one sensor in communication with said control unit.

5. The active noise attenuation system of claim 4 wherein said sensor is a tachometer.

6. The active noise attenuation system of claim 4 wherein said sensor is a throttle position sensor.

7. The active noise attenuation system of claim 4 wherein said sensor is an environmental sensor.

8. The active noise attenuation system of claim 1 wherein said speaker is disposed as part of an air induction system.

9. An air induction system comprising:
an air duct body having a speaker;
a control unit in communication with said speaker;
a memory unit in communication with said control unit storing cancellation waveform data wherein said cancellation waveform data comprises at least one cancellation waveform related with engine data.

10. The active noise attenuation system of claim 9 wherein said engine data relates to engine speed.

11. The active noise attenuation system of claim 9 further including at least one sensor in communication with said control unit.

12. The active noise attenuation system of claim 11 wherein said sensor is a tachometer.

13. The active noise attenuation system of claim 11 wherein said sensor is a throttle position sensor.

14. The active noise attenuation system of claim 11 wherein said sensor is an environmental sensor.

15. The active noise attenuation system of claim 11 wherein said speaker is disposed about an air induction system.

16. A method of attenuating noise comprising the steps of:
storing in memory at least one cancellation waveform;
retrieving the cancellation waveform needed to attenuate a noise based upon a sensed engine condition; and
attenuating the noise using the cancellation waveform.

17. The method of claim 16 wherein the noise relates to engine noise.

18. The method of claim 16 wherein the at least one cancellation waveform is related with engine speed and is retrieved and used to attenuate the noise.

19. The method of claim 16 wherein the noise is attenuated about air induction system.

20. The method of claim 16 further comprising the step of scaling the cancellation waveform.
